(19) United States
(12) Patent Application Publication
(10) Pub. No.: US 2017/0028078 A1
(43) Pub. Date: Feb. 2, 2017

(54) SUSTAINED-RELEASE DRUG FORMULATIONS FOR GLAUCOMA

(71) Applicant: LayerBio, Inc., Arlington, MA (US)

(72) Inventors: Kenneth MANDELL, Arlington, MA (US); Aleksandr WHITE, Arlington, MA (US)

(21) Appl. No.: 15/221,257

(22) Filed: Jul. 27, 2016

Related U.S. Application Data
(60) Provisional application No. 62/197,921, filed on Jul. 28, 2015.

(51) Int. Cl. A61K 47/48; A61K 31/5575; C08G 69/48; A61K 9/00

(52) U.S. Cl. CPC A61K 47/48315; A61K 9/0048; A61K 31/5575; C08G 69/48

(57) ABSTRACT

A polymer-drug conjugate includes a crosslinked polymer network comprising a biocompatible polymer and a multivalent covalent crosslinker, wherein the multivalent crosslinker comprises an active ingredient precursor covalently bonded through two or more bonds to the biocompatible polymer, and wherein the covalent bond is a hydrolysable bond. The drug can be for treatment of glaucoma and the free drug is biologically active and selected to lower eye pressure.

### RELATED APPLICATIONS

This application claims the benefit of priority under 35 U.S.C. §119(e) to copending U.S. Application Ser. No. 62/197,921 filed Jul. 28, 2015, the contents of which are incorporated by reference.

### BACKGROUND

Glaucoma is a leading cause of blindness worldwide. Blindness from glaucoma can be treated with topical eye drops, that lower eye pressure; however, many patients are not compliant with their medication regimens and continue to lose vision in spite of being prescribed such drugs. There is therefore a need for sustained-release products that automatically deliver these vital glaucoma medications and eliminate the issue of medication noncompliance.

A second issue with current glaucoma treatments is the inability to control diurnal fluctuation of eye pressure. Evidence shows that daily changes in eye pressure may be a significant factor in disease progression even if the average pressure is relatively normal. Whereas topical eye drops provide pulsatile delivery of drug to the eye that transiently lowers eye pressure, a sustained-release product that continuously delivers medication has the potential to control the diurnal fluctuations on a 24-hour basis.

### SUMMARY

The present invention provides compositions and methods related to sustained-release formulations for treatment of glaucoma and other disorders of the eye.

The present disclosure describes a drug-polymer conjugate consisting of, e.g., a glaucoma drug and, e.g., a charged and/or a water soluble polymer. The drug and polymer are connected by a hydrolysable linkage, such as an ester, amide or anhydride linkage. The drug-polymer conjugate can be formulated for use in an aqueous medium. The formulations may contain drug-polymer conjugate as a nanoparticle or microparticle. Other formulations may contain drug-polymer conjugate as part of electrostatic complexes or layer-by-layer films.

The present disclosure demonstrates that a drug spontaneously releases from the formulations under physiologic conditions, and the kinetics of drug release follow zero-order kinetics. The rate of drug release is affected by the chemical linkage between the drug and polymer. The rate of drug release is also affected by inclusion of the drug-polymer conjugate in electrostatic complexes or layer-by-layer films.

The present disclosure demonstrates that the formulations lower eye pressure. The duration of the effect of these formulations on eye pressure is longer than an ordinary glaucoma eye drop.

Methods are disclosed herein for treating ocular disorders associated with increased fluid pressure in the eye (intraocular pressure, IOP) by administering one or more of the disclosed drug formulations to a subject. The drug formulation can be administered by injection to the eye, including the subconjunctival space, anterior chamber, posterior chamber, vitreous body or suprachoroidal space. Conditions that can be treated according to the disclosed method include those characterized by increased fluid pressure in the eye, such as glaucoma or other forms of optic neuropathy.

In one aspect, a polymer-drug conjugate includes a crosslinked polymer network comprising a biocompatible polymer and a multivalent covalent crosslinker, wherein the multivalent crosslinker comprises an active ingredient precursor covalently bonded through two or more bonds to the biocompatible polymer, and wherein the covalent bond is a hydrolysable bond.

In one or more embodiments, the bond is formed with a hydroxyl, carboxylic acid, amino or mercapto moiety on the active ingredient.

In one or more embodiments, the covalent bond is an ester, amide, thioester, mercapto, carbonate, urethane, urea, anhydride, acetal, hemiacetal, ether, nitrile, phosphonate, polycyanoacrylate or anhydride bond.

In any of the preceding embodiments, the active ingredient precursor is covalently bonded to the polymer though a linker.

In any of the preceding embodiments, the biocompatible polymer comprises a charged or water soluble polymer.

In any of the preceding embodiments, the active ingredient is a glaucoma drug.

In any of the preceding embodiments, the active ingredient is a prostaglandin analog.

In any of the preceding embodiments, the active ingredient is a latanoprost, travoprost, bimatoprost, unoprostone, tafluprost, or a prodrug, derivative or metabolite of these drugs.

In any of the preceding embodiments, at least one of the polymers is a polypeptide.

In any of the preceding embodiments, the amino acids include glutamate, aspartate, lysine, arginine, and histidine.

In any of the preceding embodiments, the active ingredient is an antibiotic.

In any of the preceding embodiments, the active ingredient is chloramphenicol, erythromycin, kanamycin, vancomycin, or a prodrug, derivative or metabolite of these drugs.

In any of the preceding embodiments, the active ingredient is a corticosteroid.

In any of the preceding embodiments, the active ingredient is dexamethasone or a prodrug, derivative or metabolite of these drugs.

In any of the preceding embodiments, the drug load is in the range of about 0.1mol % to about 33mol %.

In any of the preceding embodiments, the drug load is in the range of about 1mol % to about 25mol %.

In another aspect, a method of sustained release of drug for the treatment of a condition of the eye, includes providing a drug formulation according to any preceding embodiment; and administering the drug formulation to the eye, wherein the crosslink bond hydrolyses to release the drug and treats one or more conditions of the eye.

In one or more embodiments, the condition of the eye is glaucoma, and for example, treatment of the eye includes reduction of eye pressure.

In any of the preceding embodiments, the active ingredient is a prostaglandin analog.

In any of the preceding embodiments, the active ingredient is a latanoprost, travoprost, bimatoprost, unoprostone, tafluprost, or a prodrug, derivative or metabolite of these drugs.

In any of the preceding embodiments, the condition of the eye is infection, the condition of the eye is inflammation.

In any of the preceding embodiments, the drug is released with zero-order kinetics.

In any of the preceding embodiments, the drug is released for a duration of at least 3 months.

In any of the preceding embodiments, the drug formulation is injected or implanted into the subconjunctival space, anterior chamber, posterior chamber, vitreous body or suprachoroidal space of the eye.

In any of the preceding embodiments, the drug formulation is applied outside of the eye and the drug diffuses into the eye once released from the formulation.

In any of the preceding embodiments, administration comprises injection, topical administration or implantation.

In another aspect, a pharmaceutical formulation includes a crosslinked polymer network In any of the preceding embodiments.

In one or more embodiments, the crosslinked polymer network is in the form of microparticles, nanoparticles, rods, sheets, spheres, discs and other solid drug forms.

In any of the preceding embodiments, the formulation is a suspension or dispersion.

In any of the preceding embodiments, the formulation is an implant.

In any of the preceding embodiments, the formulation is co-formulated within a matrix of another polymer.

In another aspect, a coating for or a component to a medical device for delivery of an active ingredient includes a crosslinked polymer network In any of the preceding embodiments.

In any of the preceding embodiments, the medical device is an ocular device.

In any of the preceding embodiments, the medical device is selected from the group consisting of implants, injectables, contact lenses, punctual plugs, capsular tension rings, glaucoma drainage devices, tubes, shunts, stents, sutures, pumps, corneal inlays or intraocular lenses.

In some embodiments, ranges are expressed herein as from “about” one particular value, and/or to “about” another particular value. When such a range is expressed, another embodiment includes from the one particular value and/or to the other particular value. Similarly, when values are expressed as approximations, such as by use of the antecedent “about,” it is understood that the particular value forms another embodiment. It may be further understood that the endpoints of each of the ranges are significant both in relation to the other endpoint, and independently of the other endpoint.

In this specification and in the claims which follow, reference will be made to a number of terms which shall be understood to have the following meanings:

“Optional” or “optionally” means that the subsequently described event or circumstance can but need not occur, and that the description includes instances where said event or circumstance occurs and instances where it does not.

As used herein, “subject” refers to a human or an animal; a mammalian species refers to a mammal, e.g., a human.

### BRIEF DESCRIPTION OF DRAWINGS

FIG. 1A is a schematic illustration of a 2D crosslinked drug-polymer conjugate according to one or more embodiments.

FIG. 1B is a schematic illustration of a 3D networked drug-polymer conjugate according to one or more embodiments

FIG. 2 is a table of Prostaglandin analogs used for treatment of glaucoma according to one or more embodiments.

FIG. 3 is a schematic showing a nonselective synthesis of a networked drug-polymer conjugate consisting of travoprost free acid and poly-L-glutamic acid (TPA-PGA), according to one or more embodiments.

FIG. 4 contains photographs of two different forms of TPA-PGA, “Form 1” and “Form 2”.

FIGS. 5A and 5B contain plots illustrating in vitro release profiles of TP-PGA Form 1 and Form 2, according to one or more embodiments.

FIG. 6 is a plot of the 1H-NMR spectrum of the synthesized polymer TPA-PGA according to one or more embodiments.

FIG. 7A shows the chromatograms of the UV intensity of stock travoprost free acid (TPA), stock poly-glutamic acid (PGA) and TPA-PGA Form 1 and Form 2 by HPLC according to one or more embodiments.

FIG. 7B is a plot of the UV Apex spectrum of HPLC peaks at 10.6 min for stock TPA, 6.4 min for stock PGA, and16.5 min and 20.1 min for TPA-PGA according to one or more embodiments.

FIG. 8 is a plot showing IOP lowering in beagle dogs with Form 1 TPA-PGA polymer implanted into the anterior chamber of the eye according to one or more embodiments.

FIG. 9 is a plot showing IOP lowering in beagle dogs with Form 1 TPA-PGA polymer implanted subconjunctivally according to one or more embodiments.

FIG. 10 shows the chemical structures of travoprost free acid, chloramphenicol, dexamethasone, kanamycin, erythromycin and vancomycin.

FIG. 11 shows in vitro release of drug from a selective TPA-PGA conjugate.

### DETAILED DESCRIPTION

In some embodiments of this invention, a drug delivery formulation includes a drug-polymer conjugate including a drug and a biocompatible network polymer. The polymer forms a two-dimensional polymer network or a three-dimensional polymer network with the drug. In one or more embodiments, the drug is a biodegradable crosslinker in the polymer network. In one or more embodiments, the drug-polymer network forms a nanoparticle, microparticle or macroscopic implant that can be formulated for administration to the eye.

In one or more embodiments, the polymer network includes a conjugate of a drug with the biocompatible polymer that forms degradable, e.g., hydrolysable, bonds between the polymer and the drug. The use of a drug crosslinking agent, and particularly as a crosslink agent in a 3D polymer network, tightly nests the drug within the polymer, allowing a sustained release that can occur over weeks, months, and even a year.

Non-limiting advantages arising from having the drug contribute to the structure of the network polymer include slower release rates, linear release rate, simplicity of synthesis, safety, biocompatibility and biodegradability.

Slower rate of drug release: Compared to pendant drug-polymer conjugates for which there is a single linkage per drug molecule, a drug that forms a network polymer has three or more linkages, each of which stabilizes the drug in its existing conformation and position within the molecule. For single linkages, hydrolysis is essentially an irreversible reaction, because once cleaved from the polymer, the drug can shift conformation and diffuse immediately from its previously bound site. In a network polymer, however, hydrolysis of a single linkage is not sufficient to release the drug instantaneously, because the other redundant linkages are intact. As ester hydrolysis is a reversible reaction, the drug may remain in its original position and linkages may be hydrolyzed and reform spontaneously. This results in a significantly slower release rate for a drug that forms network polymer rather than a conventional pendant drug-polymer conjugate. Furthermore, the rate of release appears to correlate with the amount of drug loading relative to the polymer.

Linear drug release profile: The current invention demonstrates zero-order drug release from a network polymer system. This is contrast with drug release from physical encapsulation systems, such as PLA or PLGA systems, which typically exhibit “burst” release at the beginning and end of the release period producing a sigmoidal release profile.

Simplicity of synthesis: The network polymer system involves a one-step synthesis in which drug is nonselectively reacted with polymer to form the network polymer. Conventional pendant drug-polymer conjugates usually require multiple steps selectively connect the drug to the polymer, and this usually involves a separate linker molecule to connect the drug and polymer. In general, fewer synthetic steps is consider an advantage in manufacturing, both for cost of goods and quality control reasons.

Biodegradation: The network polymer system is fully biodegradable. In comparison, implantable drug pumps and reservoir-based delivery systems either have to be refilled or removed and replaced, which can be a disadvantage. Furthermore, if PLGA and PLA polymers, which are biodegradable, are used to encapsulate drug for ocular drug delivery, they often degrade a slower rate than the drug is released, which results in “ghost particles” or “spent shells” that interfere with vision. So a network polymer approach results in less extraneous material left at the site of delivery than these other approaches.

Safety and biocompatibility: A network polymer comprised of only of drug and polymer does not require additional linkers apart from the drug-polymer product, and once fully degraded, all that remains are drug and monomer molecules. To the extent that the drug is an approved drug and polymer is an approved polymer with known safety, there is minimal risk of exposure to toxic byproducts resulting from degradation of the drug-polymer network. In contrast, pendant drug-polymer systems rely on linker molecules to connect the drug to the polymer, and cross-linked systems rely on linker molecules to encapsulate drug within a polymer matrix. As such, there may be greater risk of toxicity with inclusion of these linker molecules and any novel epitopes or antigens that form as result of their presence.

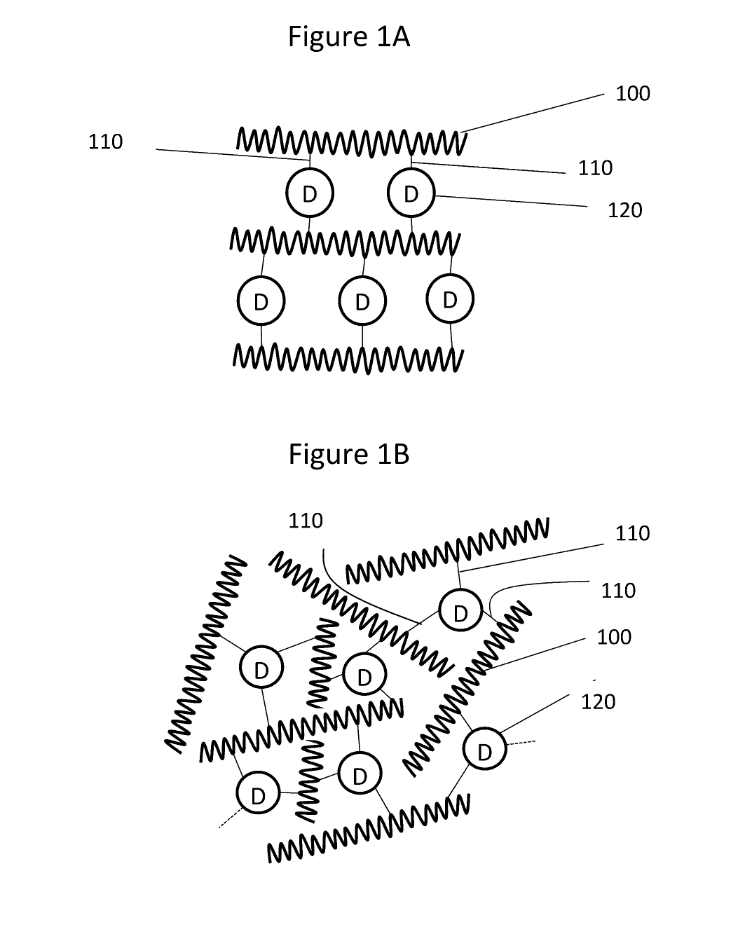

An exemplary 2D crosslinked drug-polymer conjugate is shown in FIG. 1A, while an exemplary 3D network drug-polymer conjugate is shown in FIG. 1B.

A 2D crosslinked polymer conjugate includes two linkages 110 from the drug 120 to the polymer 100 as shown in FIG. 1A. In one or more embodiments, the 2D crosslinked drug-polymer conjugate includes two linkages from the drug molecule to two different polymer molecules (intermolecular crosslinking). In other embodiments, the 2D crosslinked drug-polymer conjugate includes two linkages from the drug molecule to the same polymer molecule (intramolecular crosslinking). Note that the use of two linkages with the drug molecules does not mean or require that the drug have only two linkable or active sites. For example, a 2D network can be obtained using a higher ‘valent’ molecule by only activating two sites (as is discussed in greater detail below).

A 3D network drug-polymer conjugate includes three or more linkages 110 from the drug 120 to the polymer 100 to form a three-dimensional network of polymer connections as shown in FIG. 1B. In one or more embodiments, the 3D networked drug-polymer conjugate includes three linkages from the drug molecule to three different polymer molecules (intermolecular crosslinking). In other embodiments, the 3D networked drug-polymer conjugate includes two or more linkages from the drug molecule to the same polymer molecule (intramolecular crosslinking). Note that the use of three linkages with the drug molecules does not mean or require that the drug have only three linkable or active sites. For example, a 3D network can be obtained using a higher ‘valent’ molecule by only activating three sites (as is discussed in greater detail below). Because a number of different crosslinks can form, the resulting conjugate forms a randomly crosslinked polymer network.

Polymer 100 can be any biocompatible polymer. Non-limiting examples of biocompatible polymers include natural polymers, such as cellulose, collagen, starch blends, hyaluronic acid, alginates, carrageenan, polypeptides and the like and synthetic polymers such as silicones, polyurethanes, fluropolymers (PTFE, FRP, TEFE, PFA, MFA etc.), polycarbonate, acrylic compounds, polyesters, polyethylene and the like In one or more embodiments, the polymer should contain (or be chemically modified to contain) two or more functional groups that can form covalent hydrolysable bonds. In some embodiments, the polymer contains functional groups having hydroxyl, amino or carboxylic acid moieties that are capable of forming hydrolysable linkages 110 such as esters, ethers, amides, thioethers and thioesters and anhydrides.

Polymer 100 need not be a homopolymer and it need not be linear. It could be a complex polymer, comprised of varying ingredients connected in varying manners. This includes block polymers, cross-linked polymers, dendrimers and networked polymers.

In some embodiments, the polymer is a water soluble polymer or a charged polymer. For example, the polymer can be a positively or negatively charged polymer, such as peptides, polyamines and polycarboxylic acids. While it is not critical for the polymer to be charged, the presence of a charge can indicate the presence of a reactive site. Thus, the polymer is not required to have and may not have a charge after crosslinking. In one or more embodiments of the invention, the water soluble polymer is a peptide including charged amino acids, such as glutamine, lysine, arginine, histadine, glutamate and aspartate. In other embodiments of the invention, the water soluble polymer is polar, but not necessarily, charged such that it can form hydrogen bonds. In one specific embodiment, the polymer is polyglutamic acid or poly-L-glutamic acid (PGA), which is negatively charged polymer under physiological conditions. In one specific embodiment, the polymer is cyclodextrin polymer. Charged polymers can be alternatively referred to as polymer electrolytes.

Polymers exist in a variety of molecular weights. In general, for drug delivery systems, varying molecular weights of a given polymer may result in differing release profiles for a given drug-polymer system. Often larger polymers degrade more slowly than smaller polymers of the same composition. Larger polymer may also facilitate more chain entanglement which could also increase release rate. In some embodiments of the invention, the molecular weight of the polymer is selected to achieve a duration of drug release that is longer or shorter than a similar embodiment that utilizes the same drug and with a different molecular weight polymer.

Drug 120 can be any drug with at least two (for the formation of a 2D crosslinked drug polymer conjugate) or at least three (for the formation of a 3D network drug-polymer) functional groups that can form covalent hydrolysable bonds. In some embodiments, the drug contains three or more functional groups. In some embodiments, the drug contains three or more functional groups having hydroxyl, amino or carboxylic acid moieties that are capable of forming hydrolysable linkages 110 such as esters, ethers, amides, thioethers and thioesters and anhydrides. In one or more embodiments, the bond is formed with an ester, amide, thioester, mercapto, carbonate, urethane, urea, anhydride, acetal, hemiacetal, ether, nitrile, phosphonate or polycyanoacrylate or anhydride.

If the drug does not naturally contain three or more functional groups, it could be chemically modified to contain three or more functional groups. In such cases, it would be important for the chemically modified drug to retain similar pharmacologic properties to the parent drug.

In some embodiments a drug is conjugated to a polymer via a linker moiety. The linker moiety forms one or more of the bonds to the drug and/or charged polymer that is capable of degradation under physiological conditions. For example, the bond can be an ester or an amide linkage that hydrolytically degrades under physiologic conditions. In one or more embodiments, the crosslinked polymer network includes multiple ester bonds.

In one or more embodiments, the linker molecule includes pendant groups that are capable of chemical reaction with the glaucoma drug and/or the charged or water soluble polymer. In some embodiments the pendant groups are the same, or different. For example, the linker molecule can be triethylene glycol (TEG), having pendant hydroxyl groups, sulfydryl and/or amide groups, e.g., an ethylene glycol alcohol, thiol or amine. The hydroxyl groups are capable of reacting, for example, with organic acids or amines of the drug and/or the charged polymer to form hydrolysable bonds. In some embodiments of the invention, linker molecule has more or less ethylene glycol units than TEG, such as for example between 2 and 20 ethylene groups.

In one or more embodiment, the drug is selected for treatment of the eye. In one or more embodiment, the drug can be any drug having at least two (or at least three) functional groups capable of forming a covalent, hydrolysable bone that is currently identified or subsequently identified as suitable for treatment of glaucoma, intraocular lens pressure or other optic neuropathy can be used in accordance with the invention.

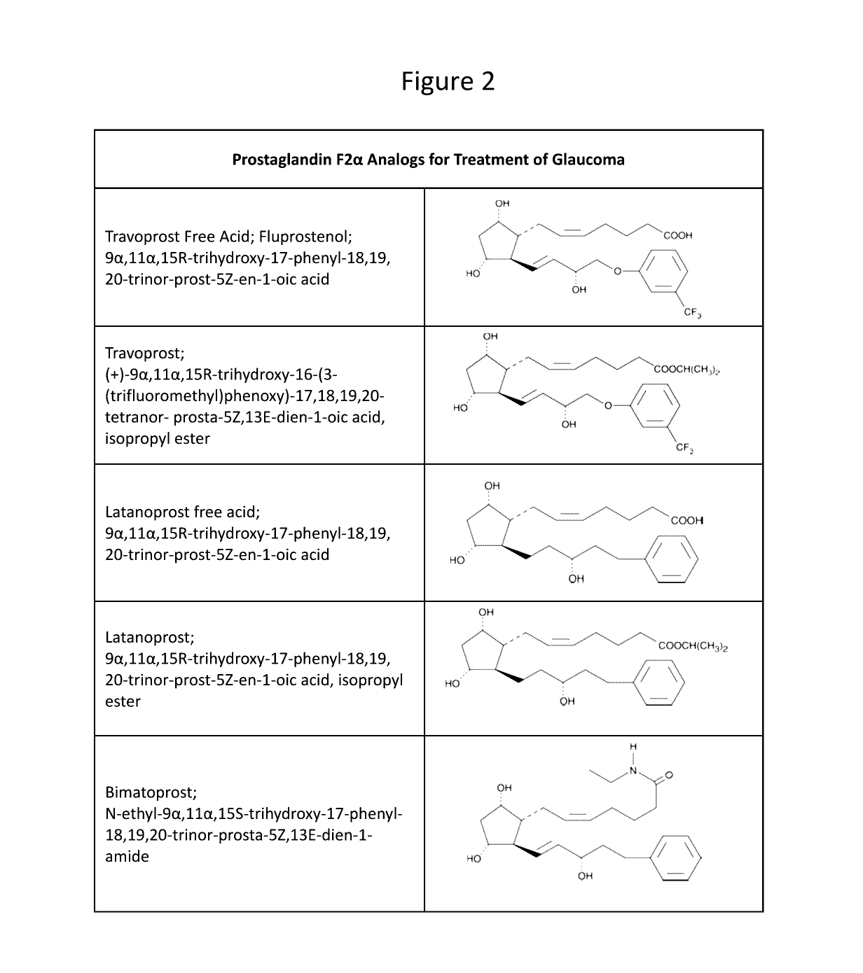

In one or more embodiments the drug is a prostaglandin analogue. Various derivatives of prostaglandin-F2α have been developed as drugs for treatment of glaucoma. As shown in FIG. 2, travoprost (TP) and latanoprost are isopropyl ester prodrugs that are naturally converted to carboxylic acids in vivo. The free acid form of travoprost is also known as fluprostenol (FP). Bimatoprost is an amide, not an ester or acid, but is otherwise similar to these drugs in structure and function. In some embodiments of the invention, the glaucoma drug is another prostaglandin analog pictured in FIG. 2.

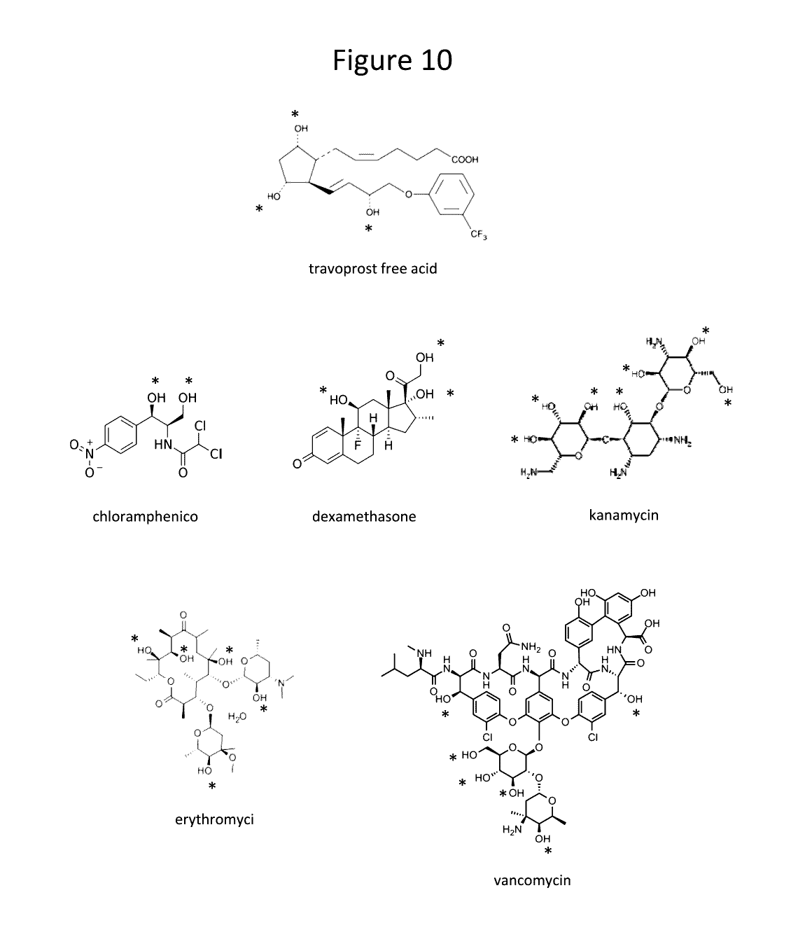

In one or more embodiments, the drug is selected from a class other than prostaglandin analogues, such as anti-inflammatory drugs, for the purpose of treating ophthalmic diseases other than glaucoma. FIG. 10 shows examples of drugs that, like TPA, are polyalcohols expected to be capable of forming cross-linked or network polymers in a manner similar to TPA via reactivity of the hydroxyl groups (labeled with asterisks).

In one or more embodiments, the free drug is a prostaglandin, beta adrenergic antagonist, alpha adrenergic agonist, carbonic anhydrase inhibitor, or muscarinic agonist.

In one or more embodiments, the free drug is acetazolamide, methazolamide, latanoprost, timolol, brimonidine, pilocarpine, dorzolamide, brinzolamide, levobunolol, echothiophate iodide, travoprost, bimatoprost, apraclonidine, metipranolol, carteolol, unoprostone, tafluprost, or a prodrug, derivative or metabolite of these drugs.

The drug load of the drug-polymer network correlates to the linkage density, e.g., the number of crosslinks per unit molecular weight, of the drug-polymer conjugate. Linkage density include crosslinks derived from drug and/or the drug/linker combination. The drug load may be affected by the hydrophobic properties of the drug. For example, it may be difficult to have a high load of a hydrophobic drug in a polymer system that is highly hydrophilic or water soluble. A high drug load provides a higher crosslink density with a corresponding effect on the solubility and hydrolysis kinetics of the drug-polymer conjugate. In one or more embodiments, high % drug loading correlated with lower water solubility and slower release of drug from the polymer. The range of drug loading can be between 5% and 50% by mass, or 2-20% by molar ratio percentage. As used herein, mol percentage refers to the fraction of drug molecules relative to the total number of molecules in the mixture expressed as a percentage, where the total number of molecules is the total number of polymer monomer units plus the total number of drug molecules. The crosslink density depends on the valence of the drug, i.e. the number of reactive groups per molecule that can form linkages with available reactive groups on the polymer. In one or more embodiments, the drug loading can vary from about 0.1 mol % to about 33 mol %. In one or more embodiments, the drug loading can vary from about 1 mol % drug to about 25 mol %. In one or more embodiments, the drug loading can vary from about 3 mol % drug to about 15 mol %.

In one or more embodiments, the drug loading can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 mol %. In other embodiments, the drug loading can be a range bounded by any value disclosed herein. This result shows that higher drug loading correlates with slower drug release from TPA-PGA formulations.

Other embodiments of the invention may include different glaucoma drugs, linkers and polymers arranged in a similar structure and imparting analogous functional properties. In addition, it is contemplated that the conjugate may include other non-drug crosslinkers, for example, to control solubility and rate of biodegradation.

In one or more embodiments, the polymer and linker ingredients are biocompatible and biodegradable and the active form of the drug is released from the prodrug, e.g., the 2D crosslinked drug-polymer conjugate or the 3D network drug-polymer conjugate, to be biologically equivalent to the original form of the drug. For example, it is acceptable to release the free acid form of travoprost (TP) (referred to as TPA), because it is biologically active and at least as potent as the isopropyl ester form of TP.

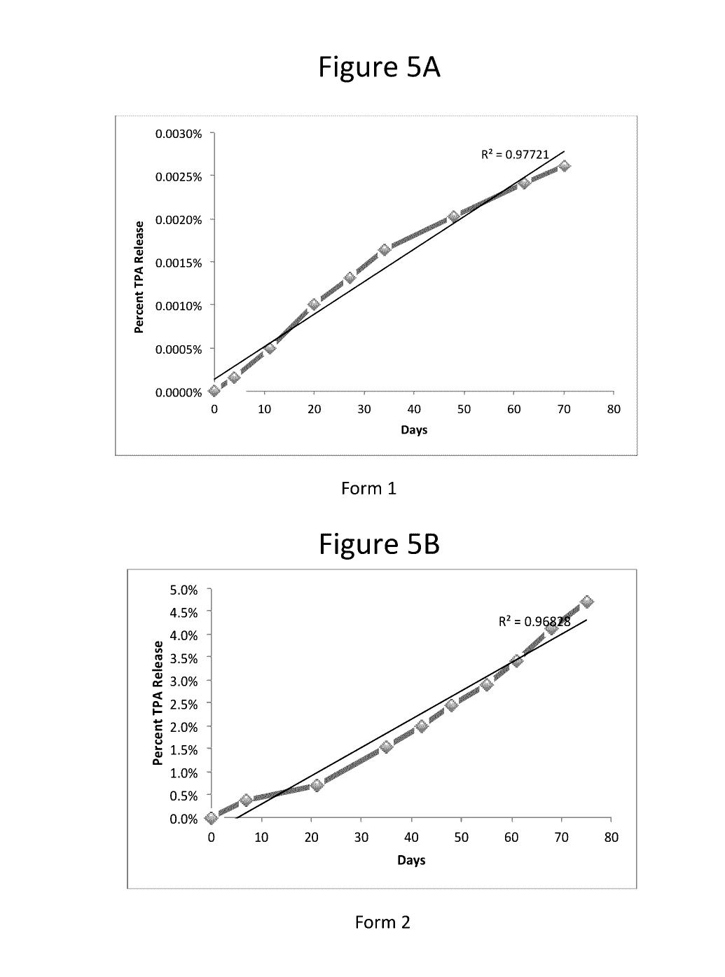

FIGS. 5A and 5B show in vitro controlled release of TPA from two different TPA-PGA (polyglutamic acid) formulations with half-lives of 3000 years and 2.5 years, respectively. After 70 days, Form 1 released about 0.0025% of total TPA, while Form 2 had released about 4.75% of total TPA. Both formulations exhibited zero-order release kinetics. The difference between the two observed releases rates may be attributed to the differences in the ratio of TPA-PGA in each formulation, where Form 1 contains more TPA per glutamic acid monomer (ca. 14 mol % TPA) than Form 2 (ca. 3.4 mol % TPA). This result shows that higher drug loading correlates with slower drug release from TPA-PGA formulations. In one or more embodiments, the drug loading can vary from about 1 mol % to about 25 mol %.

In some embodiments, the crosslinked or networked drug-polymer conjugate can be co-formulated within a matrix of another polymer. The polymeric matrix can include any polymer material useful in a body of a mammal, whether derived from a natural source or synthetic. Some additional examples of useful polymeric matrix materials for the purposes of this invention include carbohydrate based polymers such as methylcellulose, carboxymethylcellulose, hydroxymethylcellulose hydroxypropylcellulose, hydroxyethylcellulose, ethyl cellulose, dextrin, cyclodextrins, alginate, hyaluronic acid and chitosan, protein based polymers such as gelatin, collagen and glycolproteins, hydroxy acid polyesters such as poly-lactide-coglycolide (PLGA), polylactic acid (PLA), polyglycolide, polyhydroxybutyric acid, polycaprolactone, poly-valerolactone, polyphosphazene, and polyorthoesters. Other polymer carriers include albumin, polyanhydrides, polyethylene glycols, polyvinyl polyhydroxyalkyl methacrylates, pyrrolidone and polyvinyl alcohol.

In one or more embodiments, the crosslinked or networked drug-polymer conjugate can be formed as nanoparticles or microparticles.

In other embodiments, the polymer can be processed as a polymeric material into a variety of shapes, such as rods, sheets, sphere, discs and other solid drug forms.

In one or more embodiments, a pharmaceutical formulation is provided in which the polymer network is formulated to provide delivery of the active ingredient. The polymer network containing the active ingredient can be shaped or otherwise manufactured as particles or rods, and formulated in solid dosage forms or in liquid or gel dosage forms. In one or more embodiment, the polymer network can be incorporated into a pharmaceutical formulation in the form of small particles, rods, disks or other shapes. The shapes, e.g., particles or rods, can have a size, for example, a length, a width, a diameter, a cross-sectional area, a surface area, or a volume, on the order of micrometers or nanometers.

The particles or rods of the polymer network can also be combined with a pharmaceutically acceptable vehicle component in the manufacture of a pharmaceutical formulation. In other words, a pharmaceutical formulation, as disclosed herein, can include the active ingredient covalently linked as an active ingredient precursor in the polymer network, and a pharmaceutically acceptable vehicle component. In at least one embodiment, the vehicle component is aqueous-based. For example, the composition may comprise water. The aqueous vehicle component is advantageously ophthalmically acceptable and may also include one or more conventional excipients useful in ophthalmic compositions. The present pharmaceutical formulations may be, and are preferably, sterile, for example, prior to being used in the eye.

In certain embodiments, the vehicle component may also include an effective amount of at least one of a viscosity inducing component, a resuspension component, a preservative component, a tonicity component and a buffer component.

Methods of preparing these formulations include the step of bringing into association a polymer network of the present invention with a carrier and, optionally, one or more accessory ingredients. In one or more embodiments, the formulations are prepared by uniformly and intimately bringing into association a polymer network of the present invention with liquid carriers, or finely divided solid carriers, or both, and then, if necessary, shaping the product.

In other embodiments, the crosslinked or networked drug-polymer conjugate can be formulated as a coating for a medical device. In other embodiments, the polymer network may be coupled to a medical device for delivery of the active ingredient.

In some embodiments, a drug linked to a polymer may be applied to the surface of particles. The particles can be of any shape, such as spheres, ovals, rods and cones. In some embodiments of the invention, the core particle material is bioerodible, such as PLA, PLGA or chitosan.

In one or more embodiment, the networked drug-polymer conjugate could be implanted surgically or injected into the target tissue. In other embodiments, the networked drug-polymer conjugate can be applied topically as a liquid, gel, cream or ointment, or as a solid sheet or film. Other exemplary routes of delivery include oral, intraoral, intranasal, intraocular, intra-aural, dermal, subcutaneous, intradermal, intramuscular, inhalation, rectal, vaginal, urethral, intravenous, intramuscular, intraperitoneal. Dosage forms for the topical administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or butellants which may be required. The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

In one or more embodiments, the site of delivery includes any absorptive surface, physiologic compartment, solid tissue or potential space, such as the eye, ear, brain, spine, joint space, skin, muscle or circulatory system.

In particular embodiments, when the site of delivery includes the eye, the site of delivery includes the ocular surface, eyelid cul-de-sac, punctum, subconjunctival space, anterior chamber, posterior chamber, vitreous, sub-Tenon's space, orbit and suprachoroidal space. In one or more embodiments, the site of delivery is the anterior chamber of the eye. In one or more embodiments, the site of delivery is the subconjunctival space.

### EXAMPLES

The invention is explained with reference to the following examples, which are presented for the purpose of illustration only and are not intended to be limiting of the invention.

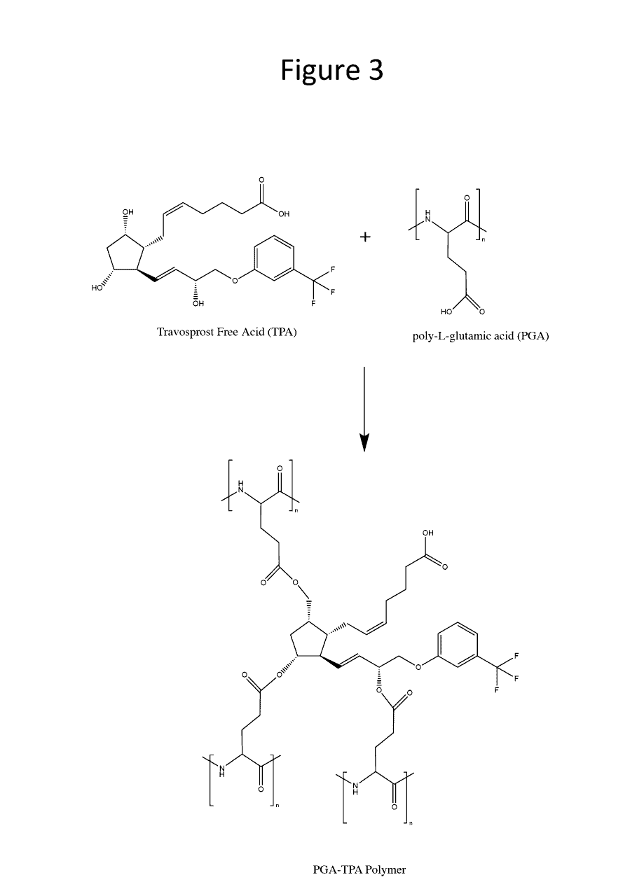

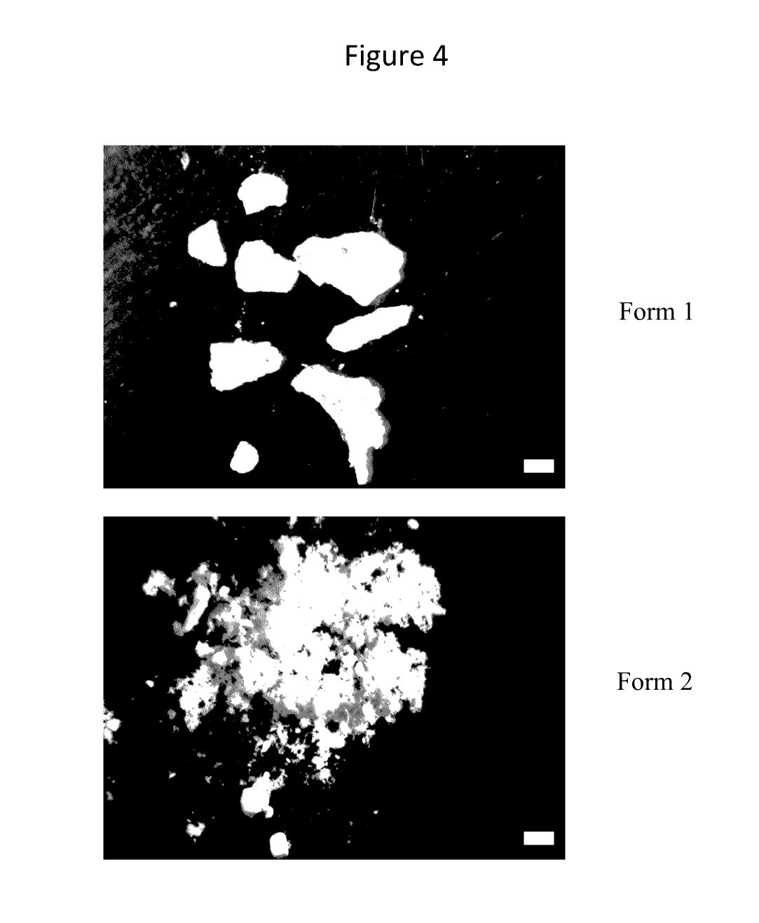

Polymer synthesis. A cross-linked polymer comprised of a drug, travoprost free acid (TPA), and a biodegradable polymer, poly glutamic acid (PGA), was synthesized using a nonselective method wherein ester bonds occur randomly between any of the three hydroxyl groups present on each TPA molecule and any of the carboxylic acid groups on PGA (FIG. 3). Non-selective conjugation of TPA to PGA was carried out via Steglich esterification. To a suspension of poly-L-glutamic acid sodium salt (Sigma, 50-75 kDa) in dimethylformamide (DMF) was added 1,3-dicyclohexylcarbodiimide (DCC). The reaction mixture was stirred at room temperature for 3 hrs. A solution of TPA (Cayman Chemical) and 4-dimethylaminopyridine (DMAP) (0.4 equiv) in DMF was added and stirred at room temperature for 48 hrs. The reaction was terminated by adding 100 mM sodium bicarbonate. The mixture was then dialyzed to remove any remaining low molecular weight materials. After dialysis, some solid precipitate was found suspended in the aqueous mix. This was filtered yielding 57.2 mg of a dense off-white solid (Form 1) and the remaining liquid was lyophilized yielding 30.9 mg of a white, fluffy solid (Form 2). Photographs of Form land Form 2 are shown in FIG. 4. Both materials were recombined in dimethyl sulfoxide (DMSO) for NMR and HPLC characterization. The material was then re-dialyzed whereupon the solid precipitate was again filtered and the liquid lyophilized.

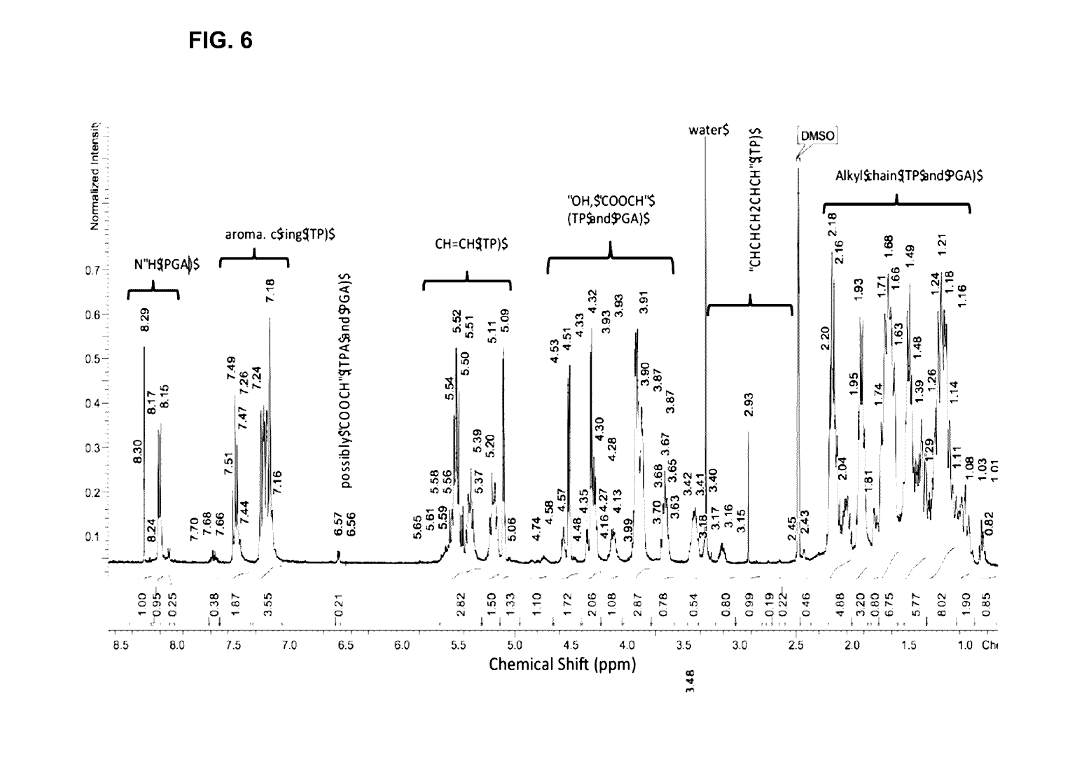

NMR Results. 1H-NMR (400 MHz, DMSO-d6) was carried out with a Varian Inova NMR (Cayman Chemical). NMR of nonselective conjugate (dissolved in DMSO) shows all of the expected TPA and PGA peaks (FIG. 6). An additional peak at 4.35 suggests the conversion of TPA alcohol group to an ester bond. In addition, the shift observed for the N—H group of unfunctionalized PGA (8.1) and that of the functionalized PGA (8.3), suggests successful conjugation of TPA to PGA.

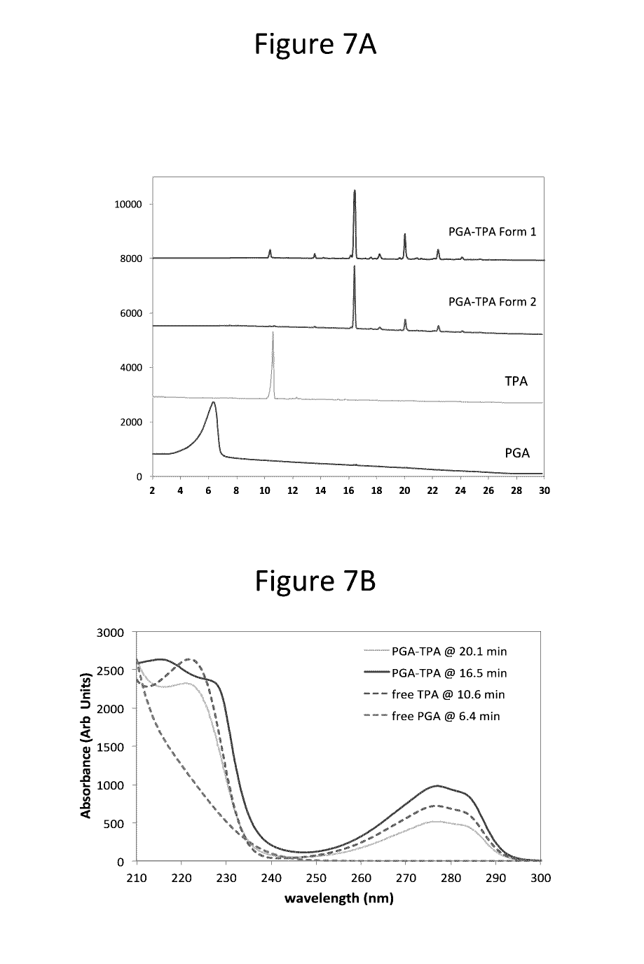

HPLC Results. reversed-phase HPLC (Agilent 1200, Cayman Chemical, Ann Arbor Mich.) was carried out with a Jupiter C5 column (300Å, 5 μm, 150×4.6mm) with 50 μL injections into a 1 ml/min mobile phase of acetonitrile/H20/trifluoroacitic acid (gradient: 20/80/0.05 to 90/10/0.05) using a variable wavelength diode array detector. HPLC chromatograms are shown in FIG. 7A.

A chromatogram of stock TPA is shown with peak at retention time of 10.6 min. The chromatogram for stock PGA reveals a broad peak at 6.4 min. The chromatogram for the nonselective TPA-PGA material shows two dominant peaks at 16.5 and 20.1 min that do not correspond to free TPA or free PGA. Furthermore, these two peaks contain the expected UV absorbance signature of TPA suggesting successful conjugation. HPLC chromatograms for Form 1 and Form 2 both exhibited the same two dominant peaks (16.5, 20.1 min), however the relative intensities differed: relative to Form 1, Form 2 peaks were 20% higher and 20% lower at 16.5 and 20.1 min, respectively. Thus a greater proportion of the Form 2 conjugate eluted from the column under a more polar mobile phase, suggesting a lower degree of TPA functionalization relative to Form 1. The presence of multiple peaks including smaller peaks ranging from 10.7 to 25.2 min (present in both Form 1 and Form 2) suggest that the material is not completely homogeneous, which is consistent with the nonselective nature of the synthesis reaction and potential for multiple bonds to occur on each TPA molecule. A

FIG. 7B is a plot of the UV absorption peaks for free PGA (dashed curves) and TPA-crosslinked PGA (solid curves).

Aqueous Solubility. Aqueous solubility of TPA-PGA was measured and results are shown in Table 1.

TABLE 1

| Polymer- | Aqueous | TPA functionalization | Half-life, t_{1/2} (mo.) |
|---|---|---|---|
| drug | solubility | with PGA | Conjugate |
| Conjugate | (mg/ml) | mol % (wt %) | prepared in PBS |
| TPA-PGA | <0.01 | 14 ± 4.1 (33 ± 7.6) | 1.4 × 10⁴ |
| (Form 1) | | | |
| TPA-PGA | ~0.05 | 3.4 ± 0.9 (9.5 ± 2.5) | 35 |
| (Form 2) | | | |

Both Forms 1 and 2 of TPA-PGA showed limited aqueous solubility, with Form 1 being highly insoluble and Form 2 being slightly soluble. The variation in solubility likely results from differing degrees of TPA conjugation and cross-linking between PGA strands, with higher TPA functionalization resulting in a more hydrophobic material. To mitigate potential solubility issues for NMR and HPLC characterization (described above), measurements were performed with dimethyl sulfoxide (DMSO) as the solvent.

Degree of TPA conjugation. To demonstrate regeneration of TPA from the polymer-drug conjugate and to determine the degree of TPA conjugation within the prodrug, samples were subjected to conditions to induce rapid ester hydrolysis as described previously. Samples added to equal volumes of DMSO and 0.1M NaOH, then sonicated for 3 minutes and left incubating overnight at 37° C. on an orbital shaker at 100 rpm. The liquid was then quenched with HC1 (of equal molar concentration to NaOH) to bring the pH to a suitable level for LCMS analysis. Quantitative LCMS (Agilent 6120, Cayman Chemical) was used to measure the concentration of TPA in solution. 50 μl samples were injected into a 0.4 mL/min mobile phase of acetonitrile/water/formic acid with gradient (10/90/0.01 to 90/10/0.1) through a Gemini C18 column (100Å, 3 μm, 50×2.0mm). Electrospray ionization (ESI) mass spectroscopy was carried out in single ion monitoring (SIM) mode for the [M-H]-anion of TPA (457.5 m/z). LCMS results confirmed the presence of free TP, demonstrating the successful recovery of the drug from the conjugate. Form 1 showed 4-fold higher degree of TPA incorporation relative to Form 2. The results are reported in Table 1.

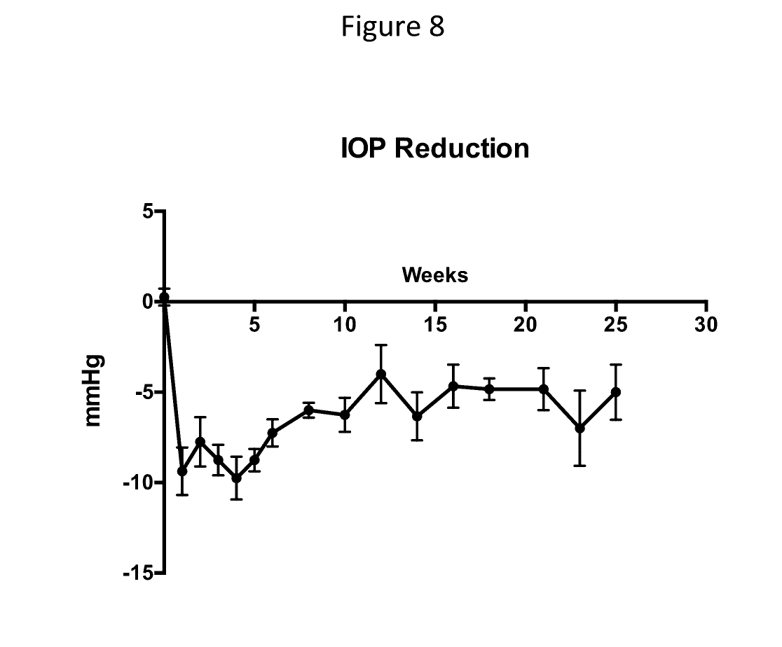

Release kinetics of drug from polymer-drug conjugate. Hydrolysis kinetics of the polymer-drug conjugate was measured by incubation of the conjugate at concentration of 0.5 mg/ml in 1× phosphate buffered saline (PBS) within a small-volume dialysis unit (Slide-A-Lyzer™ MINI; Thermo Fischer Scientific; 2K molecular weight cut-off) that was immersed in 1 ml of PBS, pH 7.4. At regular time points, 400 μl was extracted for LCMS analysis and replaced with 400 μl of fresh PBS. Elution half-lives were calculated from initial rates of TPA release measured over at least 2 to 3 weeks. Release plots are shown in FIGS. 7 and 8. Release profiles differed dramatically between Form 1 (t_{1/2} of 1.4×10⁴ days) and Form 2 (t_{1/2} of 35 days).

Intraocular pressure (IOP) lowering in beagle dogs with intracameral implants. Studies were performed to investigate the IOP lowering effects of TPA-PGA polymer in beagle dogs, a common animal model for studying glaucoma drugs. Solid implants weighing on average 2.0 mg comprised entirely of Form 1 TPA-PGA polymer were implanted into the anterior chamber of the right eye of each dog using standard ocular surgical techniques. The left eye of each animal remained untreated as a control. IOP was measured in both eyes at baseline prior to implantation of the TPA-PGA polymer into the eye and then at various time points over the next 25 weeks. As shown in FIG. 8, IOP in the treated right eye was lower than the untreated eye at all time points over the 25 week period. These results demonstrate that the TPA released from Form 1 TPA-PGA retains its biologic activity in vivo, and the duration of this effect is consistent with a sustained release mechanism via ester hydrolysis from the TPA-PGA polymer.

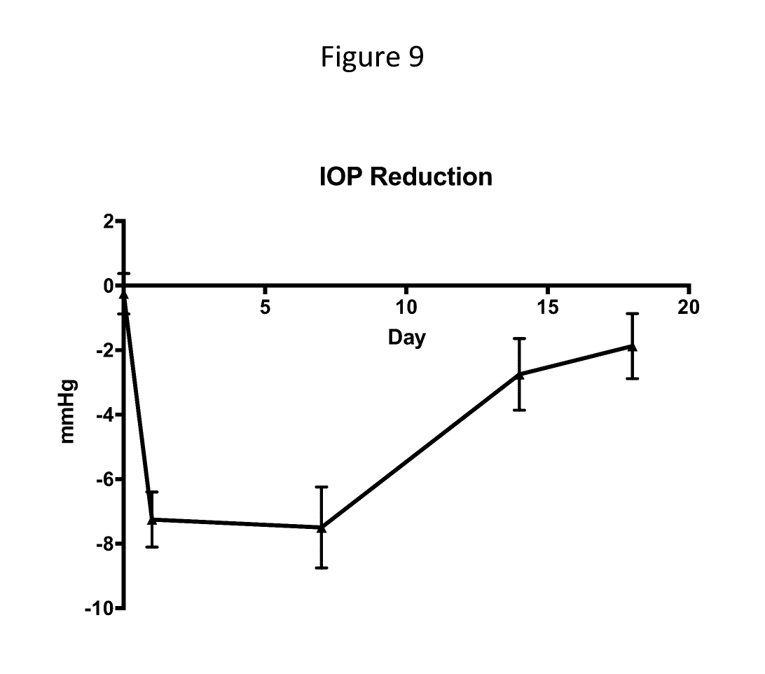

Intraocular pressure (IOP) lowering in beagle dogs with subconjunctival implants. Similar studies were performed to investigate the IOP lowering effects of TPA-PGA polymer in beagle dogs using a subconjunctival route of delivery. Solid implants weighing on average 6.5 mg comprised entirely of Form 1 TPA-PGA polymer were implanted under the conjunctiva of the right eye of each dog using standard ocular surgical techniques. The left eye of each animal remained untreated as a control. IOP was measured in both eyes at baseline prior to implantation of the TPA-PGA polymer into the eye and then at various time points over the next 4 weeks. As shown in FIG. 9, IOP in the treated right eye was significantly lower than the untreated for approximately 7 days, and then the effect wore off over the next 10 days. These results further demonstrate that the TPA released from Form 1 TPA-PGA retains its biologic activity in vivo, and the duration of this effect is consistent with a sustained release mechanism via ester hydrolysis from the TPA-PGA polymer.

### Comparative Example

The process used for non-selective crosslinking can be contrasted with the process for a selective conjugation of a drug as a single linker, e.g., an end-capped drug or a pendant drug, in that selective conjugation of a polymer-drug is carried out in three steps: Step 1. Esterification, Step 2. Deprotection and Step 3. Polymer conjugation.

Selective conjugation of the polymer-drug was carried out in three steps: Step 1. Esterification: To travoprost (120 mg, 0.26 mmol) in dichloromethane (3 mL) was added 1-(3-dimethylaminopropyl)-3-ethylcarbodiimide hydrochloride (75 mg, 0.39 mmol) and 1-hydroxybenzotriazole hydrate (72 mg, 0.53 mmol). Boc-PEG4-alcohol (750 mg, 2.56 mmol) in dichloromethane (2 mL) was added followed by addition of diisopropylethylamine (60 μL, 0.34 mmol). The reaction was stirred at room temperature overnight. Column chromatography (5:95 MeOH:CH₂Cl₂) was done to obtain the TP-PEG3-Boc product (54.9 mg) which was subsequently verified by 1H NMR. Step 2. Deprotection: To travoprost-PEG3-Boc (55 mg, 0.075 mmol) in dichloromethane (400 μL) at 0° C. was added 100 μl trifluoroacetic acid (TFA). The reaction was followed by thin layer chromatography (TLC) to determine completion. After 2 hrs at 0° C. and 2 hrs at to room temp and the addition of more TFA (100 μL), the reaction was worked up by evaporation of the solvent and TFA. Column chromatography (5:95→50:50 MeOH:CH₂Cl₂) was done to give the TFA salt of travoprost-PEG₃-NH₂ (55 mg, 100% yield). Step 3. Conjugation to PGA: To poly-L-glutamic acid sodium salt (11 mg, 0.075 mmol) in DMF (200 μL) was added DCC (8 mg, 0.039 mmol) in DMF (300 μL). The reaction was stirred for 30 min before N-hydroxysuccinimide (9 mg, 0.078 mmol) and 4-(dimethylamino)pyridine (6 mg, 0.049 mmol) were added. The reaction was stirred for 2 days at room temperature. To the reaction mixture was added TP-PEG₃-NH₂ TFA salt (55 mg, 0.075 mmol) and diisopropylethylamine (20 μL, 0.11 mmol) in DMF (400 μL). This was stirred at room temperature 72 hrs. Dialysis was done using SnakeSkin tubing (MWCO 3,500) in water to remove the lower molecular weight materials. The water was removed by lyophilization to give the final product as a white solid (19 mg). ¹H-NMR (400 MHz, DMSO-d₆) revealed —NH— amide bond of PEG to PGA at 7.86 and PEG ester bond (—COOCH2-) from TPA to PEG at 4.50 confirming conjugation via the PEG linker. In addition, TP-PEG₃-PGA chromatogram peak at 14.0 min is different from free TPA and free PGA and contains the expected UV absorbance signature of TPA suggesting successful conjugation.

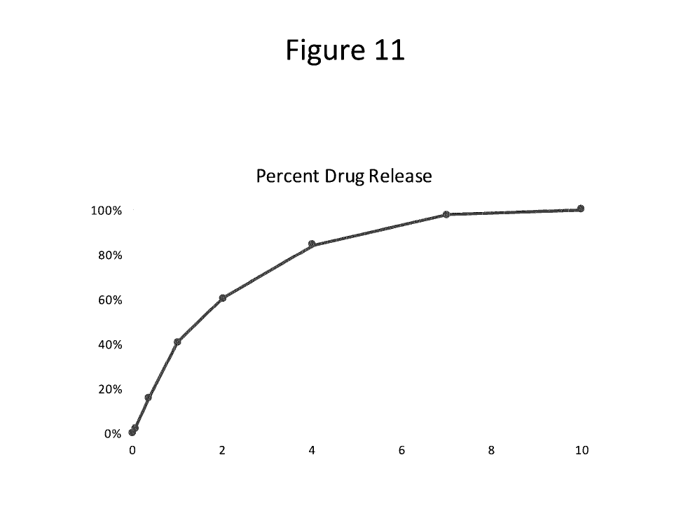

In vitro release experiments were performed with the selectively conjugated drug-polymer. As shown in FIG. 11, in contrast to TPA-PGA Forms 1 and 2, the selective conjugate did not exhibit zero-order release, and 97% of the drug released within the first 7 days. This is in contrast to similar experiments with TPA-PGA Forms 1 and 2 (FIGS. 5A and 5B) showing zero-order release for more than 70 days.

Those skilled in the art would readily appreciate that all parameters and examples described herein are meant to be exemplary and that actual parameters and examples will depend upon the specific application for which the composition and methods of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that the invention may be practiced otherwise than as specifically described. Accordingly, those skilled in the art would recognize that the use of a composition or method in the examples should not be limited as such. The present invention is directed to each individual composition, or method described herein. In addition, any combination of two or more such compositions or methods, if such composition or methods are not mutually inconsistent, is included within the scope of the present invention.

1. A polymer-drug conjugate comprising:
a crosslinked polymer network comprising a biocompatible polymer and a multivalent covalent crosslinker,
wherein the multivalent crosslinker comprises an active ingredient precursor covalently bonded through two or more bonds to the biocompatible polymer, and
wherein the covalent bond is a hydrolysable bond.

2. The polymer-drug conjugate of claim 1, wherein the bond is formed with a hydroxyl, carboxylic acid, amino or mercapto moiety on the active ingredient.

3. The polymer-drug conjugate of claim 1, wherein the covalent bond comprises an ester, amide, thioester, mercapto, carbonate, urethane, urea, anhydride, acetal, hemiacetal, ether, nitrile, phosphonate, polycyanoacrylate or anhydride bond.

4. The polymer-drug conjugate of claim 1, wherein the active ingredient precursor is covalently bonded to the polymer though a linker.

5. The polymer-drug conjugate of claim 1, wherein the biocompatible polymer comprises a charged or water soluble polymer.

6. The polymer-drug conjugate of claim 1, wherein the active ingredient comprises a glaucoma drug.

7. The polymer-drug conjugate of claim 1, wherein the active ingredient comprises a prostaglandin analog.

8. The polymer-drug conjugate of claim 1, wherein the active ingredient comprises latanoprost, travoprost, bimatoprost, unoprostone, tafluprost, or a prodrug, derivative or metabolite of these drugs.

9. The polymer-drug conjugate of claim 1, wherein at least one of the polymers comprises a polypeptide.

10. The polymer-drug conjugate of claim 9, wherein the amino acids include glutamate, aspartate, lysine, arginine, and histidine.

11. The polymer-drug conjugate of claim 1, wherein the active ingredient comprises an antibiotic.

12. The polymer-drug conjugate of claim 1, wherein the active ingredient comprises chloramphenicol, erythromycin, kanamycin, vancomycin, or a prodrug, derivative or metabolite of these drugs.

13. The polymer-drug conjugate of claim 1, wherein the active ingredient comprises a corticosteroid.

14. The polymer-drug conjugate of claim 1, wherein the active ingredient comprises dexamethasone or a prodrug, derivative or metabolite of these drugs.

15. The polymer-drug conjugate of claim 1, wherein the drug load is in the range of about 0.1 mol % to about 33 mol %.

16. The polymer-drug conjugate of claim 1, wherein the drug load is in the range of about 1 mol % to about 25 mol %.

17. A method of sustained release of drug for the treatment of a condition of the eye, comprising:
providing a drug formulation according to claim 1; and
administering the drug formulation to the eye, wherein the crosslink bond hydrolyses to release the drug and treats one or more conditions of the eye.

18. The method of claim 17, wherein the condition of the eye is glaucoma.

19. The method of claim 18, wherein treatment of the eye comprises reduction of eye pressure.

20. The method of claim 18, wherein the active ingredient comprises a prostaglandin analog.

21. The method of claim 18, wherein the active ingredient comprises latanoprost, travoprost, bimatoprost, unoprostone, tafluprost, or a prodrug, derivative or metabolite of these drugs.

22. The method of claim 17, wherein the condition of the eye comprises infection.

23. The method of claim 17, wherein the condition of the eye comprises inflammation.

24. The method of claim 17, wherein the drug is released with zero-order kinetics.

25. The method of claim 17, wherein the drug is released for a duration of at least 3 months.

26. The method of claim 17, wherein the drug formulation is injected or implanted into the subconjunctival space, anterior chamber, posterior chamber, vitreous body or suprachoroidal space of the eye.

27. The method of claim 17, wherein the drug formulation is applied outside of the eye and the drug diffuses into the eye once released from the formulation.

28. The method of claim 17, wherein administration comprises injection, topical administration or implantation.

29. A pharmaceutical formulation comprising:
a crosslinked polymer network of claim 1.

30. The formulation of claim 29, wherein the crosslinked polymer network is in the form of microparticles, nanoparticles, rods, sheets, spheres, discs and other solid drug forms.

31. The formulation of claim 30, wherein the formulation is a suspension or dispersion.

32. The formulation of claim 29, wherein the formulation is an implant.

33. The formulation of claim 32, wherein the formulation is co-formulated within a matrix of another polymer.

34. A coating for or a component to a medical device for delivery of an active ingredient, the coating or component comprising:
a crosslinked polymer network of claim 1.

35. The coating or component of claim 34, wherein the medical device is an ocular device.

36. The coating or component of claim 35, wherein the medical device is selected from the group consisting of implants, injectables, contact lenses, punctual plugs, capsular tension rings, glaucoma drainage devices, tubes, shunts, stents, sutures, pumps, corneal inlays or intraocular lenses.
